(19) United States
(12) Patent Application Publication
(10) Pub. No.: US 2009/0221656 A1
(43) Pub. Date: Sep. 3, 2009

(54) Drug Combinations

(76) Inventors: David Alexander Learmonth, Alfena (PT); Alexander Beliaev, Mindelo (PT); Patricio Manuel Vieira Araujo Soares Da Silva, Porto (PT)

Correspondence Address: CONLEY ROSE, P.C., 5601 GRANITE PARKWAY, SUITE 750, PLANO, TX, 75024, US

(21) Appl. No.: 12/160,762

(22) PCT Filed: Jan. 15, 2007
(86) PCT No.: PCT/PT07/00002

§ 371 Date: Oct. 29, 2008

(30) Foreign Application Priority Data
Jan. 13, 2006 (GB) 0600709.0

(51) Int. Cl. A61K 31/4164; A61P 9/10

(52) U.S. Cl. 514/392

(57) ABSTRACT

A combination comprising at least two components selected from:

(i) compounds of formula I:

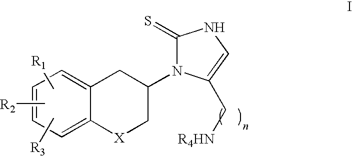

where R₁, R₂ and R₃ are the same or different and signify hydrogens, halogens, alkyl, alkyloxy, hydroxy, nitro, amino, alkylcarbonylamino, alkylamino or dialkylamino group; R₄ signifies hydrogen, alkyl or alkylaryl group; X signifies CH₂, oxygen atom or sulphur atom; n is 1, 2 or 3, with the proviso that when n is 1, X is not CH₂; and the individual (R)- and (S)-enantiomers or mixtures of enantiomers and pharmaceutically acceptable salts thereof, wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; the term halogen means fluorine, chlorine, bromine or iodine; and

(ii) at least one compound from the following classes of compounds: diuretics; beta-adrenergic antagonists; alpha2-adrenergic agonists; alpha1-adrenergic antagonists; dual beta- and alpha-adrenergic antagonists; calcium channel blockers; potassium channel activators; anti-arrhythmics; ACE inhibitors; AT1 receptor antagonists; renin inhibitors; lipid lowerers, vasopeptidase inhibitors; nitrates; endothelin antagonists; neutral endopeptidase inhibitors; anti-angiotensin vaccines; vasodilators; phosphodiesterase inhibitors; cardiac glycosides; serotonin antagonists; and CNS acting agents.

This application is a filing under 35 U.S.C. 371 of International Application No. PCT/PT2007/000002 filed Jan. 15, 2007, entitled “Drug Combinations,” claiming priority of Great Britain Patent Application No. 0600709.0 filed Jan. 13, 2006, which applications are incorporated by reference herein in their entirety.

This invention relates to drug combinations, more particularly cardiovascular drug combinations.

Compound 1 ((R)-5-(2-aminoethyl)-1-(6,8-difluorochroman-3-yl)-1,3-dihydroimidazole-2-thione hydrochloride) is a new chemical entity that integrates a series of potent inhibitors of dopamine β-hydroxylase (DBH) that were designed and synthesised incorporating modifications to the core structure of nepicastat. The aim was to provide new DBH inhibitors exerting minimal effects on dopamine (DA) and noradrenaline (NA) levels in the brain. Compound 1 is in a fact a potent and peripherally-selective DBH inhibitor. In experiments in mice and rats at Tₘₐₓ (9 h after administration), Compound 1 reduced NA levels in a dose-dependent manner in both the left atrium and the left ventricle, with the maximal inhibitory effect attained at a dose of 100 mg/kg. In contrast to that found in the heart Compound 1 failed to affect NA and DA tissue levels in brain. Compound 1 is thus presented as a candidate for clinical evaluation for the treatment of chronic heart failure and hypertension. Compound 1, together with a series of related compounds, is described in WO 2004/033447.

The rationale for the use of DBH inhibitors is based on their capacity to inhibit the biosynthesis of noradrenaline (NA), which is achieved via enzymatic hydroxylation of dopamine (DA). Activation of neurohumoral systems, chiefly the sympathetic nervous system, is the principal clinical manifestation of hypertension and congestive heart failure. Hypertensive subjects and congestive heart failure patients have elevated concentrations of plasma NA, increased central sympathetic outflow and augmented cardiorenal NA spillover. Prolonged and excessive exposure of myocardium to NA may lead to down-regulation of cardiac alpha1-adrenoceptors, remodelling of the left ventricle, arrhythmias and necrosis, all of which can diminish the functional integrity of the heart. Congestive heart failure patients who have high plasma concentrations of NA also have the most unfavourable long-term prognosis. Of greater significance is the observation that plasma NA concentrations are already elevated in asymptomatic patients with no overt heart failure, which can be used to predict ensuing mortality and morbidity. This implies that the activated sympathetic drive is not merely a clinical marker of hypertension and congestive heart failure, but may contribute to progressive worsening of both diseases.

Considering the complexity of pathophysiological mechanisms intervening in hypertension and congestive heart failure, namely the increased activity of sympathetic nervous system and increased activity of the renin-angiotensin-aldosterone system, it is of therapeutic interest to consider the combined administration of Compound 1 and drugs acting at different levels of the aforementioned systems. Because of its unique mechanism of action (selective peripheral inhibition of DBH) Compound 1 will potentiate the effects exerted by drugs acting at different levels in the sympathetic nervous system and the renin-angiotensin-aldosterone system.

Broadly, the present invention relates to drug combinations involving the following class of compounds of formula I:

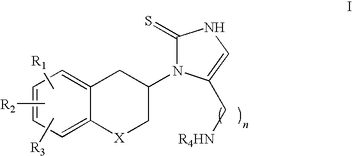

where R₁, R₂ and R₃ are the same or different and signify hydrogens, halogens, alkyl, alkylaryl, alkyloxy, hydroxy, nitro, amino, alkylcarbonylamino, alkylamino or dialkylamino group; R₄ signifies hydrogen, alkyl or alkylaryl group; X signifies CH₂, oxygen atom or sulphur atom; n is 1, 2 or 3, with the proviso that when n is 1, X is not CH₂; and the individual (R)- and (S)-enantiomers or mixtures of enantiomers and pharmaceutically acceptable salts thereof; wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; the term halogen means fluorine, chlorine, bromine or iodine. The hydrochloride salt is preferred.

More particularly, the invention relates to drug combinations involving the following specific compounds of formula I: (S)-5-(2-aminoethyl)-1-(1,2,3,4-tetrahydronaphthalen-2-yl)-1,3-dihydroimidazole-2-thione; (S)-5-(2-aminoethyl)-1-(5,7-difluoro-1,2,3,4-tetrahydronaphthalen-2-yl)-1,3-dihydroimidazole-2-thione; (R)-5-(2-aminoethyl)-1-chroman-3-yl-1,3-dihydroimidazole-2-thione; (R)-5-(2-aminoethyl)-1-(6-hydroxychroman-3-yl)-1,3 -dihydroimidazole-2-thione; (R)-5-(2-aminoethyl)-1-(8-hydroxychroman-3-yl)-1,3-dihydroimidazole-2-thione; (R)-5-(2-aminoethyl)-1-(6-methoxychroman-3-yl)-1,3-dihydroimidazole-2-thione; (R)-5-(2-aminoethyl)-1-(8-methoxychroman-3-yl)-1,3 -dihydroimidazole-2-thione; (R)-5-(2-aminoethyl)-1-(6-fluorochroman-3-yl)-1,3-dihydroimidazole-2-thione; (R)-5-(2-aminoethyl)-1-(8-fluorochroman-3-yl)-1,3-dihydroimidazole-2-thione; (R)-5-(2-aminoethyl)-1-(6,7-difluorochroman-3-yl)-1,3-dihydroimidazole-2-thione; (R)-5-(2-aminoethyl)-1-(6,8-difluorochroman-3-yl)-1,3-dihydroimidazole-2-thione; (S)-5-(2-aminoethyl)-1-(6,8-difluorochroman-3-yl)-1,3-dihydroimidazole-2-thione; (R)-5-(2-aminoethyl)-1-(6,7,8-trifluorochroman-3-yl)-1,3-dihydroimidazole-2-thione; (R)-5-(2-aminoethyl)-1-(6-chloro-8-methoxychroman-3-yl)-1,3-dihydroimidazole-2-thione; (R)-5-(2-aminoethyl)-1-(6-methoxy-8-chlorochroman-3-yl)-1,3-dihydroimidazole-2-thione; (R)-5-(2-aminoethyl)-1-(6-nitrochroman-3-yl)-1,3-dihydroimidazole-2-thione; (R)-5-(2-aminoethyl)-1-(8-nitrochroman-3-yl)-1,3 -dihydroimidazole-2-thione; (R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-aminoethyl)-1-(6-hydroxy-7-benzylchroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluorochroman-3-yl)-1,3-dihydroimidazole-2-thione; (S)-5-(3-aminopropyl)-1-(5,7-difluoro-1,2,3,4-tetrahydronaphthalen-2-yl)-1,3-dihydroimidazole-2-thione; (R,S)-5-(2-aminoethyl)-1-(6-hydroxythiochroman-3-yl)-1,3-dihydroimidazole-2-thione; (R,S)-5-(2-aminoethyl)-1-(6-methoxythiochroman-3-yl)-1,3-dihydroimidazole-2-thione; (R)-5-(2-benzylaminoethyl)-1-(6-methoxychroman-3-yl)-1,3-dihydroimidazole7-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluorochroman-3-yl)-5-(2-methylaminoethyl)-1,3-dihydroimidazole-2-thione or (R)-1-chroman-3-yl-5-(2-methylaminoethyl)-1,3-dihydroimidazole-2-thione; and pharmaceutically acceptable salts of said compounds.

More particularly, the invention relates to drug combinations involving the following specific compounds of formula I: (S)-5-(2-aminoethyl)-1-(1,2,3,4-tetrahydronaphthalen-2-yl)-1,3-dihydroimidazole-2-thione hydrochloride; (S)-5-(2-aminoethyl)-1-(5,7-difluoro-1,2,3,4-tetrahydronaphthalen-2-yl)-1,3-dihydroimidazole-2-thione hydrochloride; (R)-5-(2-aminoethyl)-1-chroman-3-yl-1,3-dihydroimidazole-2-thione hydrochloride; (R)-5-(2-aminoethyl)-1-(6-hydroxychroman-3-yl)-1,3-dihydroimidazole-2-thione hydrochloride; (R)-5-(2-aminoethyl)-1-(8-hydroxychroman-3-yl)-1,3-dihydroimidazole-2-thione hydrochloride; (R)-5-(2-aminoethyl)-1-(6-methoxychroman-3-yl)-1,3-dihydroimidazole-2-thione hydrochloride; (R)-5-(2-aminoethyl)-1-(8-methoxychroman-3-yl)-1,3-dihydroimidazole-2-thione hydrochloride; (R)-5-(2-aminoethyl)-1-(6-fluorochroman-3-yl)-1,3-dihydroimidazole-2-thione hydrochloride; (R)-5-(2-aminoethyl)-1-(8-fluorochroman-3-yl)-1,3-dihydroimidazole-2-thione hydrochloride; (R)-5-(2-aminoethyl)-1-(6,7-difluorochroman-3-yl)-1,3-dihydroimidazole-2-thione hydrochloride; (R)-5-(2-aminoethyl)-1-(6,8-difluorochroman-3-yl)-1,3-dihydroimidazole-2-thione hydrochloride; (S)-5-(2-aminoethyl)-1-(6,8-difluorochroman-3-yl)-1,3-dihydroimidazole-2-thione hydrochloride; (R)-5-(2-aminoethyl)-1-(6,7,8-trifluorochroman-3 -yl)-1,3-dihydroimidazole-2-thione hydrochloride; (R)-5-(2-aminoethyl)-1-(6-chloro-8-methoxychroman-3-yl)-1,3-dihydroimidazole-2-thione hydrochloride; (R)-5-(2-aminoethyl)-1-(6-methoxy-8-chlorochroman-3-yl)-1,3-dihydroimidazole-2-thione hydrochloride; (R)-5-(2-aminoethyl)-1-(6-nitrochroman-3-yl)-1,3-dihydroimidazole-2-thione hydrochloride; (R)-5-(2-aminoethyl)-1-(8-nitrochroman-3-yl)-1,3-dihydroimidazole-2-thione hydrochloride; (R)-5-(2-aminoethyl)-1-[6-(acetylamino)chroman-3-yl]-1,3-dihydroimidazole-2-thione hydrochloride; (R)-5-aminomethyl-1-chroman-3-yl-1,3 -dihydroimidazole-2-thione hydrochloride; (R)-5-aminomethyl-1-(6-hydroxychroman-3-yl)-1,3-dihydroimidazole-2-thione hydrochloride; (R)-5-(2-aminoethyl)-1-(6-hydroxy-7-benzylchroman-3-yl)-1,3-dihydroimidazole-2-thione hydrochloride; (R)-5-aminomethyl-1-(6,8-difluorochroman-3-yl)-1,3-dihydroimidazole-2-thione hydrochloride; (R)-5-(3-aminopropyl)-1-(6,8-difluorochroman-3-yl)-1,3-dihydroimidazole-2-thione hydrochloride; (S)-5-(3-aminopropyl)-1-(5,7-difluoro-1,2,3,4-tetrahydronaphthalen-2-yl)-1,3-dihydroimidazole-2-thione hydrochloride; (R,S)-5-(2-aminoethyl)-1-(6-hydroxythiochroman-3-yl)-1,3-dihydroimidazole-2-thione hydrochloride; (R,S)-5-(2-aminoethyl)-1-(6-methoxythiochroman-3-yl)-1,3-dihydroimidazole-2-thione hydrochloride; (R)-5-(2-benzylaminoethyl)-1-(6-methoxychroman-3-yl)-1,3-dihydroimidazole-2-thione hydrochloride; (R)-5-(2-benzylaminoethyl)-1-(6-hydroxychroman-3-yl)-1,3-dihydroimidazole-2-thione hydrochloride; (R)-1-(6-hydroxychroman-3-yl)-5-(2-methylaminoethyl)-1,3-dihydroimidazole-2-thione hydrochloride; (R)-1-(6,8-difluorochroman-3-yl)-5-(2-methylaminoethyl)-1,3-dihydroimidazole-2-thione hydrochloride or (R)-1-chroman-3-yl-5-(2-methylaminoethyl)-1,3-dihydroimidazole-2-thione hydrochloride.

Most particularly, the invention relates to drug combinations including the following specific compound of formula I: Compound 1 ((R)-5-(2-aminoethyl)-1-(6,8-difluorochroman-3-yl)-1,3-dihydroimidazole-2-thione hydrochloride). Compound 1 may be formulated with one or more compounds selected from the classes described below.

In particular the compounds of Formula I may be combined with one or more of the the following classes of compounds: diuretics; beta-adrenergic antagonists; alpha2-adrenergic agonists; alpha1-adrenergic antagonists; dual beta- and alpha-adrenergic antagonists; calcium channel blockers; potassium channel activators; anti-arrhythmics; ACE inhibitors; AT1 receptor antagonists; renin inhibitors; lipid lowerers, vasopeptidase inhibitors; nitrates; endothelin antagonists; neutral endopeptidase inhibitors; anti-angiotensin vaccines; vasodilators; phosphodiesterase inhibitors; cardiac glycosides; serotonin antagonists; and CNS acting agents.

The most useful diuretics include:
(1) Loop diuretics, in particular, furosemide, bumetanide, ethacrynic acid, torasemide, azosemide, muzolimine, piretanide, tripamide.
(2) Thiazide diuretics, in particular, bendroflumethiazole, chlorothiazide, hydrochlorothiazide, hydroflumethiazide, methylclothiazide, polythiazide, trichlormethiazide.
(3) Thiazide-like diuretics, in particular, chlorthalidone, indapamide, metozalone, quinethazone.
(4) Potassium sparing diuretics, in particular, amiloride, triamterene.
(5) Aldosterone antagonists, in particular, spirolactone, canrenone, eplerenone.
(6) Combinations of the above described diuretics.

More than one of the aforementioned diuretics may be used.

The most useful beta-adrenergic antagonists include: timolol, metoprolol, atenolol, propranolol, bisoprolol, nebivolol. More than one of the aforementioned beta-adrenergic antagonists may be used.

The most useful alpha2-adrenergic agonists include: clonidine, guanabenz, guanfacine. More than one of the aforementioned alpha2-adrenergic agonists may be used.

The most useful alpha1-adrenergic antagonists include: prazosin, doxazosin, phentolamine. More than one of the aforementioned alpha1-adrenergic antagonists may be used.

The most useful dual beta- and alpha-adrenergic antagonists include: carvedilol, labetalol. More than one of the aforementioned dual beta- and alpha-adrenergic antagonists may be used. Some of the compounds mentioned elsewhere in this application can also be used as dual beta- and alpha-adrenergic antagonists instead of, or in addition to, the compounds listed immediately above.

Potassium channel activators include nicorandil.

The most useful calcium channel blockers include: amlodipine, bepridil, diltiazem, felodipine, isradipine, nicardipine, nifedipine, nimodipine, nisoldipine, verapamil. More than one of the aforementioned calcium channel blockers may be used.

Anti-arrhythmics include: sodium channel blockers such as quinidine, procainamide, disopyramide, lidocaine, mexiletine, tocainide, phenytoin, encainide, flecainide, moricizine, and propafenone; potassium channel blockers such as: amiodarone, bretylium, ibutilide, dofetilide, azimilide, clofilium, tedisamil, sematilide, sotalol; and esmolol, propranolol, metoprolol. More than one of the anti-arrhythmics mentioned in the specification may be used. Some of the compounds mentioned elsewhere in this application can also be used as anti-arrhythmics instead of, or in addition to, the compounds listed immediately above.

The most useful ACE inhibitors include: benzepril, captopril, enalapril, fosinopril, lisinopril, imidapril, moexipril, perindopril, quinapril, ramipril, trandolapril. More than one of the aforementioned ACE inhibitors may be used.

The most useful AT1 receptor antagonists include: candesartan, irbesartan, losartan, telmisartan, valsartan, eprosartan. More than one of the aforementioned AT1 receptor antagonists may be used.

Lipid lowerers include: statins such as atorvastatin, cerivastatin, fluvastatin, lovastatin, mevastatin, pitavastatin, pravastatin, rosuvastatin, simvastatin; bile acid sequestrants such as cholestyramine, colestipol and colesevelam; cholesterol absorption inhibitors such as ezetimibe; fibrates such as fenofibrate, gemfibrozil; niacin. More than one of the aforementioned lipid lowerers may be used.

The most useful nitrates include, organic nitrates such as: amyl nitrite, nitroglycerin, isosorbide dinitrate, isosorbide-5-mononitrate, erythrityl tetranitrate. More than one of the aforementioned organic nitrates may be used.

Endothelin antagonists include: bosentan, sitaxsentan. More than one of the aforementioned endothelin antagonists may be used.

The most useful vasodilators include: hydralazine, minoxidil, sodium nitroprusside, diazoxide. More than one of the aforementioned vasodilators may be used. Some of the compounds mentioned elsewhere in this application can also be used as vasodilators instead of, or in addition to, the compounds listed immediately above.

The most useful phosphodiesterase inhibitors include: milrinone, inamrinone. More than one of the aforementioned phosphodiesterase inhibitors may be used.

Cardiac glycosides include: allocar, corramedan, digitoxin, digoxin, lanoxin, purgoxin, cedilanid-D, crystodigin, lanoxicaps. More than one of the aforementioned cardiac glycosides may be used.

Serotonin antagonists include: clozapine, loxapine, olanzapine, risperidone, ziprasidone, ritanserin, ketanserin, amoxapine. More than one of the aforementioned serotonin antagonists may be used.

CNS acting agents include imidazoline agonists such as moxonidine. The most useful CNS acting agent is methyldopa. Some of the compounds mentioned elsewhere in this application can also be used as CNS acting agents instead of, or in addition to, the compounds listed immediately above.

The most useful renin inhibitors include: aliskiren, enalkiren, ditekiren, terlakiren, remikiren, zankiren, ciprokiren. More than one of the aforementioned renin inhibitors may be used.

The most useful vasopeptidase inhibitors include: omapatrilat, sampatrilat, gemopatrilat. More than one of the aforementioned vasopeptidase inhibitors may be used.

Other pharmaceuticals used in treating heart failure may also be combined with Compound 1. These include calcium sensitisers; HMG CoA reductase inhibitors; vasopressin antagonists; adenosine A1 receptor antagonists; atrial natriuretic peptide (ANP) agonists; chelating agents; corticotrophin-releasing factor receptor; glucagon-like peptide-1 agonists; sodium, potassium ATPase inhibitors; advanced glycosylation end-products (AGE) crosslink breakers; mixed neprilysin/endotheliin-converting enzyme (NEP/ECE) inhibitors; nociceptin receptor (ORL-1) agonists (e.g. alprazolam); xanthine oxidase inhibitors; benzodiazepine agonists; cardiac myosin activators; chymase inhibitors; endothelial nitric oxide synthase (ENOS) transcription enhancers; neutral endopeptidase inhibitors such as thiorphan.

The invention also envisages the use of nepicastat with the classes of compounds described above.

Thus the invention envisages the combination of the compounds of formula I with the additional compounds described above. The combinations can be formulated into a pharmaceutical composition, optionally with at least one pharmaceutically acceptable carrier. The pharmaceutical formulations may take any appropriate form, including oral compositions, such as tablets, capsules, powders and suspensions.

The invention also relates to a method of treating disease including the step of administering a therapeutically effective amount of one of the combinations described above using to a subject in need thereof.

Diseases and disorders which may be usefully treated by using a combination of the invention include but are not limited to the following: hypertension; heart failure such as chronic or congestive heart failure; angina; arrhythmias; circulatory disorders such as Raynaulds phenomenon; migraine and anxiety disorders.

As used herein, the term treatment and variations such as ‘treat’ or ‘treating’ refer to any regime that can benefit a human or non-human animal. Thus the treatment may be in respect of an existing condition or may be prophylactic (preventative treatment). Treatment may include curative, alleviation or prophylactic effects.

According to another aspect of the invention there is provided the use of a combination as described above in the manufacture of a medicament for treating disorders where a reduction in the hydroxylation of dopamine to noradrenaline is of therapeutic benefit.

According to another aspect of the invention there is provided the use of a combination as described above in the manufacture of a medicament for treating a subject afflicted by cardiovascular disorders.

According to another aspect of the invention there is provided the use of a combination as described above in the manufacture of a medicament for treating hypertension or chronic heart failure.

According to another aspect of the invention there is provided the use of a combination as described above in the manufacture of a medicament for use in inhibiting dopamine-β-hydroxylase.

The invention also relates to a pharmaceutical package comprising a combination as described above together with instructions for simultaneous, separate or sequential use thereof. The instructions may described the use in any of the above mentioned therapies.

It will be appreciated that the invention may be modified within the scope of the claims.

1. A combination comprising at least two components selected from:
(i) compounds of formula I: 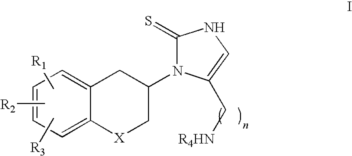
where R₁, R₂ and R₃ are the same or different and signify hydrogens, halogens, alkyl, alkylaryl, alkyloxy, hydroxy, nitro, amino, alkylcarbonylamino, alkylamino or dialkylamino group; R₄ signifies hydrogen, alkyl or alkylaryl group; X signifies CH₂, oxygen atom or sulphur atom; n is 1, 2 or 3, with the proviso that when n is 1, X is not CH₂; and the individual (R)- and (S)-enantiomers or mixtures of enantiomers and pharmaceutically acceptable salts thereof; wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; the term halogen means fluorine, chlorine, bromine or iodine;
(ii) at least one compound from the following classes of compounds: diuretics; beta-adrenergic antagonists; alpha2-adrenergic agonists; alpha1-adrenergic antagonists; dual beta- and alpha-adrenergic antagonists; calcium channel blockers; potassium channel activators; anti-arrhythmics; ACE inhibitors; ATI receptor antagonists; renin inhibitors; lipid lowerers, vasopeptidase inhibitors; nitrates; endothelin antagonists; neutral endopeptidase inhibitors; anti-angiotensin vaccines; vasodilators; phosphodiesterase inhibitors; cardiac glycosides; serotonin antagonists; and CNS acting agents; and
(iii) optionally, at least one pharmaceutically acceptable carrier, wherein said combination of (i) and (ii) are formulated for simultaneous, separate or sequential use.

2-39. (canceled)

40. The combination according to claim 1, wherein the compound of formula I is (S)-5-(2-aminoethyl)-1-(1,2,3,4-tetrahydronaphthalen-2-yl)-1,3-dihydroimidazole-2-thione; (S)-5-(2-aminoethyl)-1-(5,7-difluoro-1,2,3,4-tetrahydronaphthalen-2-yl)-1,3-dihydroimidazole-2-thione; (R)-5-(2-aminoethyl)-1-chroman-3-yl-1,3-dihydroimidazole-2-thione; (R)-5-(2-aminoethyl)-1-(6-hydroxychroman-3-yl)-1,3-dihydroimidazole-2-thione; (R)-5-(2-aminoethyl)-1-(8-hydroxychroman-3-yl)-1,3-dihydroimidazole-2-thione; (R)-5-(2-aminoethyl)-1-(6-methoxychroman-3-yl)-1,3-dihydroimidazole-2-thione; (R)-5-(2-aminoethyl)-1-(8-methoxychroman-3-yl)-1,3-dihydroimidazole-2-thione; (R)-5-(2-aminoethyl)-1-(6-fluorochroman-3-yl)-1,3-dihydroimidazole-2-thione; (R)-5-(2-aminoethyl)-1-(8-fluorochroman-3-yl)-1,3-dihydroimidazole-2-thione; (R)-5-(2-aminoethyl)-1-(6,7-difluorochroman-3-yl)-1,3-dihydroimidazole-2-thione; (R)-5-(2-aminoethyl)-1-(6,8-difluorochroman-3-yl)-1,3-dihydroimidazole-2-thione; (S)-5-(2-aminoethyl)-1-(6,8-difluorochroman-3-yl)-1,3-dihydroimidazole-2-thione; (R)-5-(2-aminoethyl)-1-(6,7,8-trifluorochroman-3-yl)-1,3-dihydroimidazole-2-thione; (R)-5-(2-aminoethyl)-1-(6-cbloro-8-methoxychroman-3-yl)-1,3-dihydroimidazole-2-thione; (R)-5-(2-aminoethyl)-1-(6-methoxy-8-chlorochroman-3-yl)-1,3-dihydroimidazole-2-thione; (R)-5-(2-aminoethyl)-1-(6-nitrochroman-3-yl)-1,3-dihydroimidazole-2-thione; (R)-5-(2-aminoethyl)-1-(8-nitrochroman-3-yl)-1,3-dihydroimidazole-2-thione; (R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-aminoethyl)-1-(6-hydroxy-7-benzylchroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluorochroman-3-yl)-1,3-dihydroimidazole-2-thione; (S)-5-(3-aminopropyl)-1-(5,7-difluoro-1,2,3,4-tetrahydronaphthalen-2-yl)-1,3-dihydroimidazole-2-thione; (R,S)-5-(2-aminoethyl)-1-(6-hydroxythiochroman-3-yl)-1,3 -dihydroimidazole-2-thione; (R,S)-5-(2-aminoethyl)-1-(6-methoxythiochroman-3-yl)-1,3-dihydroimidazole-2-thione; (R)-5-(2-benzylaminoethyl)-1-(6-methoxychroman-3-yl)-1,3-dihydroimidazole-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione ; (R)-1-(6,8-difluorochroman-3-yl)-5-(2-methylaminoethyl)-1,3-dihydroimidazole-2-thione or (R)-1-chroman-3-yl-(2-methylaminoethyl)-1,3-dihydroimidazole-2-thione; or a pharmaceutically acceptable salt thereof.

41. The combination according to claim 1, wherein the compound of formula I is (S)-5-(2-aminoethyl)-1-(1,2,3,4-tetrahydronaphthalen-2-yl)-1,3-dihydroimidazole-2-thione hydrochloride; (S)-5-(2-aminoethyl)-1-(5,7-difluoro-1,2,3,4-tetrahydronaphthalen-2-yl)-1,3-dihydroimidazole-2-thione hydrochloride; (R)-5-(2-aminoethyl)-1-chroman-3-yl-1,3-dihydroimidazole-2-thione hydrochloride; (R)-5-(2-aminoethyl)-1-(6-hydroxychroman-3-yl)-1,3-dihydroimidazole-2-thione hydrochloride; (R)-5-(2-aminoethyl)-1-(8-hydroxychroman-3-yl)-1,3-dihydroimidazote-2-thione hydrochloride; (R)-5-(2-aminoethyl)-1-(6-methoxychroman-3-yl)-1,3-dihydroimidazole-2-thione hydrochloride; (R)-5-(2-aminoethyl)-1-(8-methoxychroman-3-yl)-1,3-dihydroimidazole-2-thione hydrochloride; (R)-5-(2-aminoethyl)-1-(6-fluorochroman-3-yl)-1,3-dihydroimidazole-2-thione hydrochloride; (R)-5-(2-aminoethyl)-1-(8-fluorochroman-3-yl)-1,3-dihydroimidazole-2-thione hydrochloride; (R)-5-(2-aminoethyl)-1-(6,7-difluorochroman-3-yl)-1,3-dihydroimidazole-2-thione hydrochloride; (R)-5-(2-aminoethyl)-1-(6,8-difluorochroman-3-yl)-1,3-dihydroimidazole-2-thione hydrochloride; (S)-5-(2-aminoethyl)-1-(6,8-difluorochroman-3-yl)-1,3-dihydroimidazole-2-thione hydrochloride; (R)-5-(2-aminoethyl)-1-(6,7,8-trifluorochroman-3-yl)-1,3-dihydroimidazole-2-thione hydrochloride; (R)-5-(2-aminoethyl)-1-(6-chloro-8-methoxychroman-3-yl)-1,3-dihydroimidazole-2-thione hydrochloride; (R)-5-(2-aminoethyl)-1-(6-methoxy-8-chlorochroman-3-yl)-1,3-dihydroimidazole-2-thione hydrochloride; (R)-5-(2-aminoethyl)-1-(6-nitrochroman-3-yl)-1,3-dihydroimidazole-2-thione hydrochloride; (R)-5-(2-aminoethyl)-1-(8-nitrochroman-3-yl)-1,3-dihydroimidazole-2-thione hydrochloride; (R)-5-(2-aminoethyl)-1-[6-(acetylamino)chroman-3-yl]-1,3-dihydroimidazole-2-thione hydrochloride; (R)-5-aminomethyl-1-chroman-3-yl-1,3-dihydroimidazole-2-thione hydrochloride; (R)-5-aminomethyl-1-(6-hydroxychroman-3-yl)-1,3-dihydroimidazole-2-thione hydrochloride; (R)-5-(2-aminoethyl)-1-(6-hydroxy-7-benzylchroman-3-yl)-1,3-dihydroimidazole-2-thione hydrochloride; (R)-5-aminomethyl-1-(6,8-difluorochroman-3-yl)-1,3-dihydroimidazole-2-thione hydrochloride; (R)-5-(3-aminopropyl)-1-(6,8-difluorochroman-3-yl)-1,3-dihydroimidazole-2-thione hydrochloride; (S)-5-(3-aminopropyl)-1-(5,7-difluoro-1,2,3,4-tetrahydronaphthalen-2-yl)-1,3-dihydroimidazole-2-thione hydrochloride; (R,S)-5-(2-aminoethyl)-1-(6-hydroxythiochroman-3-yl)-1,3-dihydroimidazole-2-thione hydrochloride; (R,S)-5-(2-aminoethyl)-1-(6-methoxythiochroman-3-yl)-1,3-dihydroimidazole-2-thione hydrochloride; (R)-5-(2-benzylaminoethyl)-1-(6-methoxychroman-3-yl)-1,3-dihydroimidazole-2-thione hydrochloride; (R)-5-(2-benzylaminoethyl)-1-(6-hydroxychroman-3-yl)-1,3-dihydroimidazole-2-thione hydrochloride; (R)-1-(6-hydroxychroman-3-yl)-5-(2-methylaminoethyl)-1,3-dihydroimidazole-2-thione hydrochloride; (R)-1-(6,8-difluorochroman-3-yl)-5-(2-methylaminoethyl)-1,3-dihydroimidazole-2-thione hydrochloride or (R)-1-chroman-3-yl-5-(2-methylaminoethyl)-1,3-dihydroimidazole-2-thione hydrochloride.

42. The combination according to claim 1, wherein the compound of formula I is ((R)-5-(2-aminoethyl)-1-(6,8-difluorochroman-3-yl)-1,3-dihydroimidazole-2-thione hydrochloride).

43. The combination according to claim 1, wherein the diuretic is a loop diuretic.

44. The combination according to claim 43, wherein the loop diuretic is furosemide, bumetanide, ethacrynic acid, torasemide, azosemide, muzolimine, piretanide and/or tripamide.

45. The combination according to claim 1, wherein the diuretic is a thiazide diuretic.

46. The combination according to claim 45, wherein the thiazide diuretic is bendroflumethiazole, chlorothiazide, hydrochlorothiazide, hydroflumethiazide, methylclothiazide, polythiazide and/or trichlormethiazide.

47. The combination according to claim 1, wherein the diuretic is a thiazide-like diuretic.

48. The combination according to claim 47, wherein the thiazide-like diuretic is chlorthalidone, indapamide, metozalone and/or quinethazone.

49. The combination according to claim 1, wherein the diuretic is a potassium sparing diuretic.

50. The combination according to claim 49, wherein the potassium sparing diuretic is amiloride and/or triamterene.

51. The combination according to claim 1, wherein the diuretic is an aldosterone antagonist.

52. The combination according to claim 51, wherein the aldosterone antagonist is spirolactone, canrenone and/or eplerenone.

53. The combination according to claim 1, wherein the beta-adrenergic antagonist is: timolol, metoprotol, atenolol propranolol, bisoprolol and/or nebivolol.

54. The combination according to claim 1, wherein the alpha2-adrenergic agonist is: clonidine, guanabenz and/or guanfacine.

55. The combination according to claim 1, wherein the alpha1-adrencrgic antagonist is: prazosin, doxazosin and/or phentolamine.

56. The combination according to claim 1, wherein the dual beta- and alpha-adrenergic antagonists is carvedilol and/or labetalol.

57. The combination according to claim 1, wherein the potassium channel activator is nicorandil.

58. The combination according to claim 1, wherein the calcium channel blocker is: amlodipine, bepridil, diltiazem, felodipine, isradipine, nicardipine, nifedipine, nimodipine, nisoldipine and/or verapamil.

59. The combination according to claim 1, wherein the anti-arrhythmic is: a potassium channel blocker such as: amiodarone, bretylium, ibutilide, dofetilide, azimilide, clofilium, tedisarnil, sematilide, and sotalol; quinidine, procainamide, disopyramide, lidocaine, mexiletine, tocainide, phenytoin, encainide, flecainide, moricizine, propafenone, esmolol, propranolol, and/or metoprolol.

60. The combination according to claim 1, wherein the ACE inhibitor is: benzepril, captopril, enalapril, fosinopril, lisinopril, imidapril, moexipril, perindopril, quinapril, ramipril and/or trandolapril.

61. The combination according to claim 1, wherein the AT1 receptor antagonist is: candesartan, irbesartan, losartan, telmisartan, valsartan and/or eprosartan.

62. The combination according to claim 1, wherein the lipid lowerer is: a statin such as atorvastatin, cerivastatin, fluvastatin, lovastatin, mevastatin, pitavastatin, pravastatin, rosuvastatin, and simvastatin; a bile acid sequestrant such as cholestyramine, colestipol and colesevelam; a cholesterol absorption inhibitor such as ezetimibe; a fibrate such as fenofibrate and gemfibrozil; and/or niacin.

63. The combination according to claim 1, wherein the nitrate is: amyl nitrite, nitroglycerin, isosorbide dinitrate, isosorbide-5-mononitrate and/or erythrityl tetranitrate.

64. The combination according to claim 1, wherein the endothelin antagonist is: bosentan and/or sitaxsentan.

65. The combination according to claim 1, wherein the vasodilator is: hydralazine, minoxidil, sodium nitroprusside and/or diazoxide.

66. The combination according to claim 1, wherein the phospdiesterase inhibitor is: milrinone and/or inamrinone.

67. The combination according to claim 1, wherein the cardiac glycoside is: allocar, corramedan, digitoxin, digoxin, lanoxin, purgoxin, cedilanid-D, crystodigin, and/or lanoxicaps.

68. The combination according to claim 1, wherein the serotonin antagonist is: clozapine, loxapine, olanzapine, risperidone, ziprasidone, ritanserin, ketanserin, and/or amoxapine.

69. The combination according to claim 1, wherein the CNS acting agent is moxonidine and/or methyldopa.

70. The combination according to claim 1, wherein the renin inhibitor is: aliskiren, enalkiren, ditekiren, terlakiren, remikiren, zankiren and/or ciprokiren.

71. The combination according to claim 1, wherein the vasopeptidase inhibitor is: omapatrilat, sampatrilat and/or gemopatrilat.

72. A method comprising using a combination according to claim 1 in the manufacture of a medicament for treating disorders where a reduction in the hydroxylation of dopamine to noradrenaline is of therapeutic benefit.

73. A method comprising using a combination according to claim 1 in the manufacture of a medicament for treating a subject afflicted by cardiovascular disorders.

74. A method comprising using a combination according to claim 1 in the manufacture of a medicament for treating hypertension or chronic heart failure.

75. A method comprising using a combination according to claim 1 in the manufacture of medicament for treating one or more of the following indications: angina, arrhythmias, circulatory disorders, migraine and anxiety disorders.

76. A method comprising using a combination according to claim 1 in the manufacture of a medicament for use in inhibiting dopamine-β-hydroxylase.

77. A commercial package comprising a combination according to claim 1 together with instructions for simultaneous, separate or sequential use thereof.
